(19) United States
(12) Patent Application Publication
(10) Pub. No.: US 2004/0102810 A1
(43) Pub. Date: May 27, 2004

(54) Hyperspace torque generator

(76) Inventor: John Quincy St. Clair, San Juan, PR (US)

Correspondence Address: John St. Clair, Hyperspace Research Institute, 52 Kings Court, 4A, San Juan, PR, 00911, US

(21) Appl. No.: 10/304,260

(22) Filed: Nov. 26, 2002

(51) Int. Cl. A61N 1/00

(52) U.S. Cl. 607/1

(57) ABSTRACT

A hyperspace torque generator which comprises a flat bifilar magnetic bucking field electrical coil, crystal ball, lever arm and frequency generator for producing a linear flow of hyperspace energy at right angles to a rotating flow of astral chakra energy.

### BRIEF SUMMARY OF THE INVENTION

This invention generates a body torque using a crystal ball mounted on a cylindrical stand above a bifilar electrical coil. The coil is driven by a ten-volt sine wave frequency generator. The bipolar rotating hand chakras are placed across the ball in order to condense the astral energy of the aura. The magnetic bucking field of the coil flows low density hyperspace energy up through the ball. The torque generated is equal to the lever arm times the linear momentum of the hyperspace energy crossed with the angular velocity of the hand chakras. The device is used to demonstrate the existence of co-dimensional hyperspace energy and to show the nature of the human hyperspace energy being.

### BACKGROUND OF THE INVENTION

A human being is a hyperspace energy being residing in a physical body. Our energy is located in astral space has which has a very low velocity of light. In our four-dimensional spacetime universe where the physical body is located, the speed of light is enormous, equal to 299792458 meters per second. In astral space, on the other hand, the speed of light is much lower, resulting in a low density (0.03 to 0.07 kg) energy being that has the ability to move through solid concrete walls as well as interpenetrate the watery human body and control its movements.

This interpenetration takes the form of energy vortices that funnel energy into several co-dimensions of astral space. Over the lifetime of the individual, this energy creates the aura of the human astral being. The aura, in turn, provides protection for the organs of the body. As shown in FIG. 1, the human body has seven major vortices, known as chakras, located along the length of the body in addition to a vortex in each hand. The chakras are (A) the crown or top chakra through which energy (information) can be channeled from other energy beings, (B) the spiritual eye which allows us to see when out of the physical body, (C) the throat chakra which allows verbal communication with other beings, (D) the heart chakra which protects the cardiovascular system, (E) abdomen chakra which protects the digestive system, (F) the soul chakra which is related to the creation of newborn astral entities during fertilization, and (G) the ground chakra which grounds the energy system to earth, similar to an electrical ground. The soul is actually an orange-colored plasma energy ball which acts as a battery for the body when the astral being is out of body. It is located behind the soul chakra.

There is a vortex in each hand (H). This vortex can be readily observed by placing the right hand face up on a table. The left hand holds a pendulum over the palm of the right hand. The pendulum will start to rotate counter-clockwise over the right hand. Over the left hand, the vortex rotates clockwise. They rotate in opposite directions. Placing the hands together in the prayer position then creates a rotating cylindrical flow of energy between the hands as shown in FIG. 2. The energy (C) flows out of the right hand (B) in a counterclockwise motion (D) and enters the left hand (A).

As seen in FIG. 3, the astral energy (A) is rotating, according to the right-hand rule of physics, with the fingers pointing in the direction of the arrow and the thumb pointing to the left. Thus the angular momentum vector (C) points to the left.

Physicist John A. Wheeler of Princeton University has made it clear that there is a second term in Newton's gravitational formula as seen below:

F=−mg+m(v×Ω)

The downward force F or weight of the body is equal to the mass (m) of the body times the acceleration of gravity (g). If the body is rotating, the force is either more or less depending on the direction of the angular velocity (Ω) which is crossed with the linear velocity v of the mass. As an example of this, notice that basketball player Michael Jordan is able to float more than the other players. As he moves down the court and jumps with the ball in his hands, he accelerates the chakra energy between the hands and resonates the energy inside the spherical ball. His forward velocity then becomes crossed with the rotating chakra energy which provides him sufficient extra lift to out jump his opponents. It was also learned that Jordan clears out the locker room just before a game and meditates. This allows him time to concentrate on increasing the spin of his chakra energy.

Experimentation with flat bifilar magnetic bucking coils has shown that this type of coil can create a flow of low density hyperspace energy up through the center of the coil. This creates a linear momentum of hyperspace energy which is in the second term of the equation. Crossing this linear momentum with the angular momentum of the hand chakras located a lever arm above the coil in a crystal ball produces the hyperspace torque.

### SUMMARY OF THE INVENTION

It is the object of this invention to produce a body torque using a crystal ball mounted on a fixed lever arm above a flat bifilar magnetic field bucking coil. The coil creates a small wormhole into hyperspace which allows low density and hence low velocity speed of light hyperspace energy to enter into our four-dimensional spacetime universe. By placing the hands on either side of the ball, rotating astral energy flows from the right-hand chakra to the left-hand chakra. The linear momentum of the hyperspace energy crossed with the angular momentum of the astral energy times the lever arm offset above the coil generates an oscillating body torque. If the crystal ball is placed on the coil, no torque is generated because the lever arm is zero. Because the oscillation occurs in both space and hyperspace, the torque oscillates for nine seconds and then stops oscillating for the same period, whereupon it continues in this periodic fashion.

### STATEMENT REGARDING FEDERALLY SPONSORED RESEARCH OR DEVELOPMENT

Not Applicable.

### A Brief Description of the Drawings

FIG. 1. Chakra vortices of the human body.

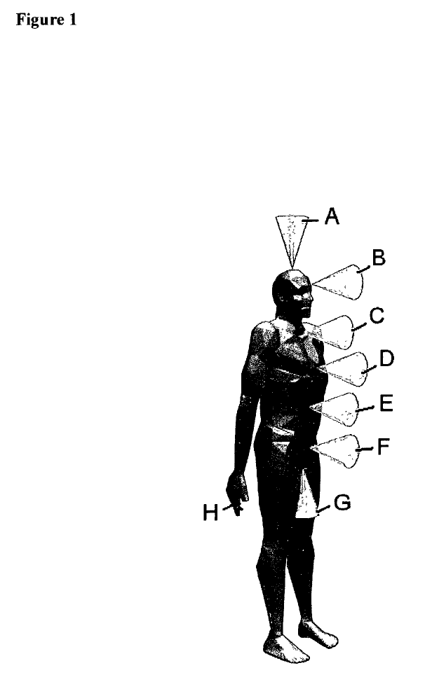

FIG. 2. Rotating chakra energy field between hands.

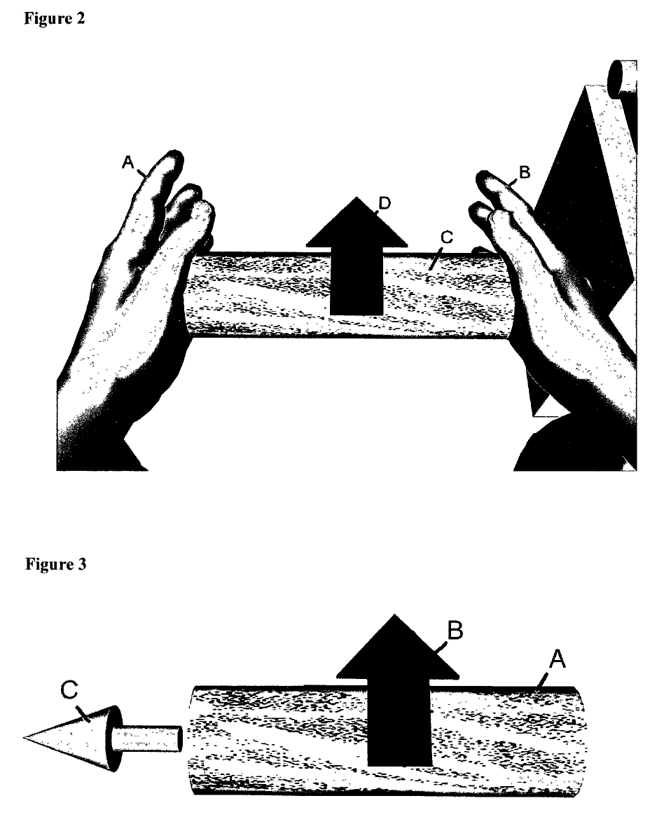

FIG. 3. Angular momentum vector of rotating energy field.

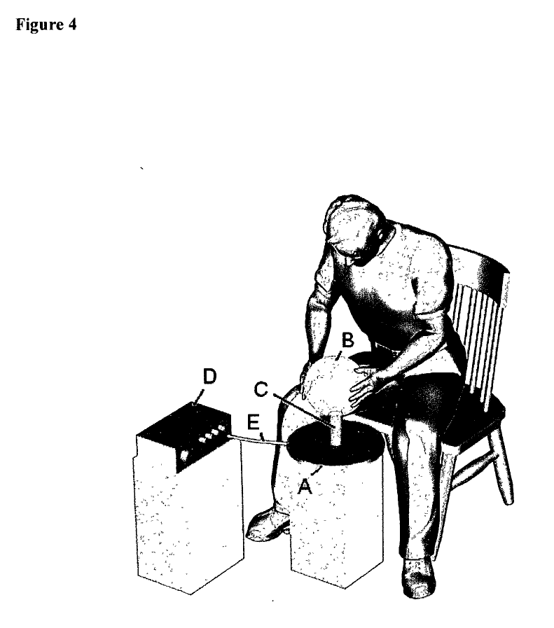

FIG. 4. Hyperspace torque generator.

### DETAILED DESCRIPTION OF THE INVENTION

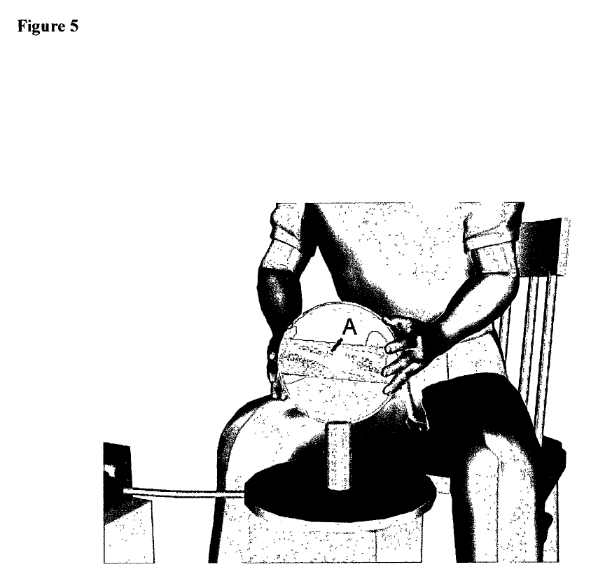

The hyperspace torque generator is shown in FIG. 4. A circular bifilar coil (A) and wooden board form the base of the unit with a crystal ball (B) mounted on a lever arm (C) attached to the center of the base. The bifilar coil is made by taking a length of insulated wire and then doubling it in half. Starting near the center of the base, the two wires of the coil are wound together in a circular manner on the flat base and then glued to the wooden base. The double wire on the inside is brought out from the center of the coil, with one side serving as one lead to the frequency generator (D). The other inner wire is connected to a wire on the outside so that the electricity flows in the opposite direction in each succeeding coil. Due to Maxwell's law, this creates a magnetic bucking field down the center of the coil which opens up hyperspace and allows energy to flow into our dimension. This energy can actually be felt as it rises through the feet. The remaining length of wire on the outer wrap forms the second terminal for the frequency generator. Both these wires are fed to the frequency generator (E).

FIG. 5 shows a close-up of the hand chakra energy (A) flowing between the hands through the crystal ball. In this configuration, the body rocks forward and backward in an oscillating manner for nine seconds. If the hands are rotated ninety degrees such that one hand is on the outside of the ball and the other is on the inside of the ball, the body rocks sideways left and right according to the right hand rule of physics.

I claim:

1. A hyperspace torque generator comprising:
a. flat bifilar magnetic bucking field electrical coil mounted on wooden base;
b. lever arm attached to wooden base and concentric with coil;
c. crystal ball attached to top of lever arm;
d. frequency generator for driving coil.
